(19) United States
(12) Patent Application Publication
(10) Pub. No.: US 2003/0049207 A1
(43) Pub. Date: Mar. 13, 2003

(54) Clinical method for evaluating the efficacy of caries prevention or treatment agents

(75) Inventors: Aaron Reed Biesbrock, Maineville, OH (US); Robert Daniel Bartizek, Lebanon, OH (US)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(73) Assignee: THE PROCTER & GAMBLE COMPANY

(21) Appl. No.: 09/949,939

(22) Filed: Sep. 10, 2001

(51) Int. Cl. A61B 5/00; A61K 49/00

(52) U.S. Cl. 424/9.2; 600/300

(57) ABSTRACT

A method of conducting clinical trials to evaluate the efficacy of agents or methods of caries prevention and treatment comprising the steps of conducting a visual-tactile assessment on qualifying human subjects of moderate to high caries incidence populations, assigning subjects to control group(s) and experimental group(s), supervising tooth brushing one or more times per day for a period of about 8 to about 12 months which runs concurrently with the school year and thereafter evaluating the efficacy of the product.

### FIELD OF THE INVENTION

The present invention relates to the field of methods of conducting clinical trials to evaluate agents or methods of caries prevention or treatment. In particular this invention relates to a clinical trial method for evaluating the efficacy of caries preventative agents.

### BACKGROUND OF THE INVENTION

Clinical research is a component of medical and health research intended to produce knowledge valuable for understanding human disease, preventing and treating illness, and promoting health. One of the primary avenues of conducting clinical research is the clinical trial. A clinical trial is, essentially, any form of planned experiment that involves patients and is designed to elucidate the most effective treatment for patients with a given medical or health condition. Clinical trials are typically utilized to evaluate the safety or effectiveness of a new treatment, medication or device. The real value of the clinical trial is that results derived from a limited sample of test subjects lead to inferences about how treatments should be conducted in the general population of patients in the future. In the dental caries field, the clinical trial is considered the fundamental method of population-based research.

While caries rates have been on the decline across a variety of geographies in the past 40 years, worldwide caries remains a prevalent disease that is still the primary cause of tooth loss. Fluoride, delivered through community water fluoridation and fluoridated dentifrices, has been shown to be the most cost effective public health mechanism for preventing tooth decay. However, access to fluoride remains a problem worldwide. In the United States, where fluoridation of community drinking water was instituted in 1945, a significant proportion of the population, as high as 40%, still does not have access to drinking water with optimal levels of fluoride. Thus, there remains a need for improved caries prevention or treatment products and treatment regimens. In turn, caries clinical trials are necessary to test the safety and efficacy of these new or improved caries prevention or treatment products that are being produced.

Historically, clinical studies measuring the prevention of caries have been conducted over a two or three year period and have involved high numbers of study participants in order to achieve significant results. High costs are associated with such large and lengthy clinical trials with costs averaging around $1 million for each year the study is on-going.

Efforts in the recent past have been made to reduce costs associated with these caries clinical trials by shortening the study duration. Unilever Research recently conducted a 12-month study to demonstrate feasibility of a short-term study for purposes of determining the differences in the anticariogenic efficacy of dentifrices. See R. K. Chesters, et al., Proof-of-Principle Abbreviated Clinical Caries Trial: 12-month outcome, J. Dent. Res. 80 (IADR Abstracts) 2001. The study encompassed 2387 Lithuanian school children divided into one test group and one control group. Clinical visual assessment (CVA) methods were used prior to, and upon completion of, the 12-month period of dentifrice use to evaluate tooth surfaces as either D₁, indicating white spot or incipient lesions, or D₃, indicating clinically detectable open or closed lesions in dentine. No significant intergroup differences were noted at the conventional D₃ threshold. Significant differences in caries incidence between the test groups were noted only with relation to the occurrence of incipient or white spot lesions, or at the D₁ threshold. It was noted that differentiation between normal and high efficacy dentifrices could be achieved after 12 months of product use based on the D₁ threshold variations detected. However, variations at the D₁ threshold are insufficient to show anti-caries efficacy of a new product based on guidelines set by the American Dental Association (ADA).

The ADA guidelines for evaluating the anti-caries efficacy of a fluoride-containing dentifrice state that tooth surfaces characterized as “decayed” must have evidence of a clinically relevant carious lesion. This means that variations at the D₁ threshold are insufficient to show anti-caries efficacy. The so-called “clinical caries” (D₂ threshold and beyond) must be used utilized as the definitive marker of disease. Incipient lesions or white spot lesions, as measured in the Unilever trial, are used as secondary markers of disease only.

Thus, there remains a need for an inexpensive, short-duration caries clinical trial method that is able to show anti-caries efficacy of an anti-caries oral care product such as a fluoride-containing dentifrice while complying with the guidelines set forth by the ADA for efficacy evaluation.

### SUMMARY OF THE INVENTION

The present invention relates to a clinical trial method for evaluating the anticaries efficacy of caries prevention or treatment products comprising the following steps. First, conducting a visual-tactile caries assessment of the tooth surfaces of qualifying human subjects living in populations with a moderate to high incidence of caries. Second, randomly assigning qualifying human subjects to two or more test groups wherein at least one group receives an experimental caries prevention or treatment product and at lease one group receives a control product. Third, supervising the human subjects while brushing their teeth with the provided products one or more times per day for a period of about 8 months to about 12 months wherein the period of supervised brushing runs concurrently with and is limited to the school year. Finally, comparing the efficacy of the caries prevention or treatment product against the efficacy of the control product by a visual-tactile caries assessment of all subjects. Typically, the human subjects are between about 5 to about 15 years old, in one embodiment, the human subjects are between about 9 and about 12 years old. Each test group can be comprised of from about 100 to about 500 people and each test group within the same clinical trial can be of approximately equal size.

### DETAILED DESCRIPTION OF THE INVENTION

The present invention relates to a clinical trial method for evaluating the anticaries efficacy of caries prevention or treatment products comprising the steps of:

a) conducting visual-tactile caries assessment on qualifying human subjects living in populations which exhibit a moderate to high incidence of caries,

b) randomly assigning qualifying human subjects to two or more test groups, wherein at least one group is an experimental group that is provided an experimental caries prevention or treatment product and at least one group is a control group that is provided a control product,

c) supervising the human subjects brushing their teeth with the assigned control product one or more times per day for a period of about 8 to about 12 months which runs concurrently with and is limited to the school year, and

d) comparing the efficacy of the experimental caries prevention or treatment product to the efficacy of the control product through a follow-up visualtactile caries assessment of all human subjects upon completion of the period of supervised brushing,

wherein throughout the period of supervised brushing, restorative dentistry performed on the human subjects is controlled.

The present invention relates to a clinical method for evaluating the anticaries efficacy of experimental caries prevention or treatment products. The clinical method of the present invention can be used to show superior efficacy of one or more experimental caries prevention or treatment products as compared to no treatment at all. Alternatively, the clinical method can be used to show efficacy of one or more experimental caries prevention or treatment products as compared to a known standard, for example, a dentifrice containing 1100 ppm of fluoride ion. Additionally, the clinical method can also be used to demonstrate that an experimental caries prevention or treatment product has anti-caries efficacy that is equal to a known standard such as a dentifrice containing 1100 ppm of fluoride ion.

Definitions

The term “caries” as used herein relates to dental caries or tooth decay. Dental caries are the progressive decalcification of the enamel and dentin of a tooth.

A “visual-tactile caries assessment” is an examination performed on the oral cavity, and specifically the tooth or tooth surfaces of an individual to determine the integrity of the tooth or tooth surface. The examination comprises a visual assessment to locate white spot or open lesions and a tactile assessment with the use of dental implements or explorers to locate decayed, softened or “sticky” lesions on the teeth or tooth surfaces. Where a strict visual caries assessment is conducted, no tactile evaluation is performed. In the instant invention, a visual-tactile caries assessment is performed both prior to and upon completion of the period of supervised brushing. DMFS or DMFT are the typical scoring methods used in the visual-tactile caries assessments.

The term “DMFS” as used herein is indicative of the scoring method used to evaluate the tooth surface in the visual-tactile caries assessments of the human subjects, which occur both prior to and after the period of supervised brushing. In this scoring method, each non-sound tooth surface receives a score of either “D”, which indicates decay, “M”, which indicates missing, or “F”, which indicates filled. The “S”, stands for (tooth) surface. A score of “D” may be further classified as D₁, D₂, D₃ and D₄ each indicating a different diagnostic threshold that relates to the severity of dental caries. D₁ denotes clinically detectable enamel lesions with intact surfaces. D₂ denotes clinically detectable “cavities” limited to enamel. D₃ denotes clinical detectable lesions in dentine, open or closed. D₄ denotes lesions extending into the pulp chamber. In accordance with ADA guidelines, surfaces scored as decayed must have evidence of having a clinically relevant carious lesion. Only surfaces demonstrating visible and/or tactile loss of tooth structure are considered to reflect a clinically relevant carious lesion. Thus, in the instant invention, only lesions characterized by D₂, D₃ or D₄ are used to identify a tooth surface as decayed. In the present invention, surfaces that show evidence of demineralization in the absence of a tactile change denoting loss of enamel structure, D₁ will be scored as sound (including incipient lesions, white spot lesions and hypomineralized intact surfaces). In DMFS scoring one score is given for each tooth surface. Five surfaces per posterior tooth, specifically buccal, lingual, occlusal, mesial and distal, and four surfaces per anterior tooth, specifically buccal, lingual, mesial and distal, are scored. A total DMFS score is given to each subject, the number indicating the total number of tooth surfaces that are decayed, missing or filled.

The term “DMFT” as used herein is an alternate scoring method that may be employed when visual-tactile assessments are conducted of the teeth. In DMFT scoring, “D”, “M” and “F” indicate decayed, missing and filled, respectively. The “T” indicates tooth. In the instant invention teeth are further categorized as D₁, D₂, D₃ and D₄ as described above. In DMFT scoring only one score is given for each whole tooth, rather than each individual tooth surface being scored independently. The total DMFT score given to each subject reflects the total number of teeth that are decayed, missing or filled.

The term “clinically relevant carious lesion” as used herein means a lesion on the tooth or tooth surface that is categorized as D₂, D₃ or D₄ as described above.

The term “DMFS increment” or “DMFT increment” as used herein means the difference in total DMFS or DMFT scores determined before and after the period of supervised brushing.

By the term “caries prevention or treatment product” or “test product” as used herein is meant any product with caries efficacy potential, for example, any product which is not intentionally swallowed for purposes of systematic administration of anti-caries agents, but is retained in the oral cavity for a sufficient time to contact all of the dental surfaces an/or oral mucosal tissues for purposes of oral activity. The product may be in any desired form, including but not limited to, paste, gel, liquid, powder and the like where the product is applied to the tooth surface through brushing. In one embodiment the caries prevention or treatment product is a dentifrice product in toothpaste form that contains a fluoride ion source. Typically there is an unknown benefit or aspect, such as the anti-caries efficacy of the test product, that the clinical trial is designed to measure or determine.

The term “control product” as used herein means a product wherein the anticaries efficacy is known and can provide a baseline against which to compare the anti-caries efficacy of the test product. In one embodiment of the present invention, the control product may be a placebo product. It has been found that in a controlled, short term, (8-12 month) clinical caries trials, where the test subjects are engaging in supervised brushing at least two times per day, a benefit is delivered to the dental surfaces of the test subjects by virtue of the supervised brushing alone. The use of a dentifrice product imparts a benefit above that of the mechanical cleaning. Therefore, the use of a placebo product as a control product is appropriate in the present invention. In another embodiment the control product(s) contain a level of fluoride ion which has been demonstrated to be clinically effective, including but not limited to, 250 ppm, 500 ppm, 1000 ppm, 1100 ppm, 1500 ppm, 2500 ppm, 2800 ppm, and 5000 ppm. The fluoride ion source may be delivered from a any safe and effective source, including but not limited to, sodium fluoride, mono fluoro phosphate, potassium fluoride, stannous fluoride, and ammonium fluoride.

The term “placebo product” as used herein relates to a product that does not contain an oral care active and is typically provided in the same product form as the test product.

The term “restorative dentistry” as used herein means any treatment, material or device that restores a tooth surface, or replaces a tooth or all teeth and adjacent tissue.

The term “qualifying human subjects” or “test subjects” as used herein refers to human children that are typically between the ages of about 5 to about 15 years of age, in one embodiment between the ages of about 8 to about 13 years of age, in another embodiment between the ages of about 9 and about 12 years of age. To qualify for inclusion in the clinical trial the subjects must be in good general health. Human subjects wearing orthodontic appliances or those who anticipate being fitted for orthodontic appliances at any time during the study period are not considered qualifying human subjects. In one embodiment, human subjects with little or no caries experience may be excluded. For example, an eight year old child with no history of caries activity in primary teeth may be excluded from the study.

The term “experimental group” as used herein is meant to indicate those qualifying human subjects that are part of the group that is provided with the experimental caries preventative product. In the present invention one or more experimental groups are present within a single clinical trial.

The term “control group” as used herein indicates those qualifying human subjects that are part of the group that is provided the control product.

The term “moderate to high caries rate population” as used herein means a population that resides in a geography which lacks a fluoridated water supply, where there is infrequent access to fluoridated dentifrices and where there is a relatively high average sugar intake per person. Populations with these characteristics are typically identified through published data and literature. In one embodiment, a population wherein the average DMFS increment is above about 0.7 per year is considered a moderate to high caries rate population. This means that within the population, an average increase of 0.7 more tooth surfaces are scored as either decayed, missing or filled after one year.

The Clinical Method

Initially, an appropriate subject pool must be located from which human subjects are chosen for participation in the clinical caries trial. In establishing a subject pool, it is important to identify and screen potential test subjects living in a population that has a moderate to high incidence of caries. Where a clinical caries trial is conducted in a population of low or low to moderate caries incidence the control group may have such a low increment of caries that it is virtually impossible to statistically demonstrate a treatment advantage. Populations of moderate to high caries rates are used to facilitate detection of variations in caries rate increment.

Once a population of moderate to high caries incidence has been identified, potential subjects are screened to exclude individuals that are wearing orthodontic appliances at the start of the clinical study. Additionally, any individual that anticipates being fitted with orthodontic appliances at any time during the study period is excluded from the study group. The test subjects must also be of good general health.

A baseline assessment is conducted on those remaining test subjects living within the identified population. The baseline visit may consist of collection of demographic data as well as completion of a visual-tactile caries assessment. One or more examiners may be used to perform the visual-tactile assessments of the qualifying human subjects. Training of the examiners, as well as periodic recalibration of the assessment method may be required to ensure accurate and consistent results and to promote the importance of objective assessments. The results of the initial visual-tactile assessment, in the form of DMFS or DMFT scores, are used as a baseline to determine caries activity in all qualifying test subjects over the study period. In one embodiment, the visual-tactile assessment may be supplemented by appropriate bitewing radiographs. The number of bitewing films will vary from about zero to about four per subject depending on the number of permanent teeth and the openness of the embrasures. In another embodiment the visual-tactile assessment may be supplemented with fiber optic transillumination (FOTI) or dual intensity fiber optic transillumination (DIFOTI). All information is stored until completion of the clinical trial.

Once the baseline visual-tactile caries assessments are complete, the randomized, double blind study begins. In a randomized double blind study, qualifying human subjects are randomly assigned to test groups comprising a control group and one or more experimental groups. In one embodiment, test subjects residing in the same households are assigned to the same test group to reduce error in product use. Neither the study participant, nor the study administrator is aware of which test subjects belong which test group until completion of the study duration and the final visual-tactile caries assessments. Each test group will contain between about 50 and about 500 qualifying human subjects, in one embodiment between about 100 and about 300 qualifying human subjects. Each test group within a single clinical trial should be of a comparable size. In one embodiment the human subjects are from about age 5 to about age 15 at the start of the clinical trial, in another embodiment the subjects are from about age 8 to about age 13 at the start of the clinical trial, and in yet another embodiment the subjects are from about age 9 to about age 12 at the start of the clinical trial.

Prior to the initial supervised brushing session, the control group or groups will be provided a control product for use throughout the entire period of supervised brushing. In one embodiment the control product is a placebo product. The experimental group or groups will each be provided an experimental caries preventative product. Test subjects should be given brushing instructions prior to the initial supervised brushing session. In one embodiment test subjects are instructed to brush, while ensuring to over all tooth surfaces, for at least about 30 seconds per session. In another embodiment the brushing session will last for at least about 60 seconds. Longer supervised brushing sessions, from about 1 minute to about 5 minutes, may be employed in alternate embodiments of the invention.

The supervised brushing regimen will commence as soon as possible after the baseline assessments are completed and test subjects have received their assigned products. The test subjects will engage in supervised brushing sessions at least once per day, in one embodiment supervised brushing sessions are held at least twice per day. The supervised brushing sessions are held during the regular course of the school day. A trained supervisor oversees the brushing sessions. Test subjects may be broken into smaller groups to facilitate supervision.

Throughout the period of evaluation and supervised brushing any restorative dentistry performed on the test subjects is controlled. Restorative dentistry may be controlled in several ways, including but not limited to, initially selecting a study population with minimal restoration intervention historically, supplying the restorative dentists that will complete the dental work so that a comprehensive record of any restoration performed is kept, or by a random balancing of the restoration to be performed among those restorative dentists already within the community.

The duration of the supervised brushing segment of the clinical trial method will correspond with the school year observed within the chosen geography. As the duration of the supervised brushing segment of the clinical trial method runs concurrent with the school year, unknowns associated with an extended summer vacation are avoided. A higher level of control is maintained throughout the study period. Discrepancies between reported brushing and actual brushing which may occur as a result of extended unsupervised periods of brushing are avoided. In one embodiment the supervised brushing sessions continue for a period of about 8 to about 12 months, in another embodiment for a period of about 9 to about 11 months.

Test subjects may be supplied with additional product which corresponds with the subject's assigned test group. This additional product may be used outside of the regular supervised brushing sessions, including, but not limited to, in the evening, on weekends and on school holidays.

Upon completion of the supervised brushing segment of the clinical trial method, the second visual-tactile assessment of all test subjects is performed. In one embodiment, the visual-tactile assessment may be supplemented by appropriate bitewing radiographs. The number of bitewing films will vary from about zero to about four per subject depending on the number of permanent teeth and the openness of the embrasures. Caries increment rates, which represent the change in DMFS or DMFT scores from the start of the supervised brushing period to the final visualtactile caries assessment, are compiled. A comparison of caries increment rates for each test group is conducted using appropriate, standard accepted statistical analysis of DMFS and DMFT increment. In one embodiment, treatment group differences are assessed using an analysis of covariance on DMFS increment scores, with baseline DMFS scores as one of the covariates.

Although the above description contains many specificities, these should not be construed as limitations on the scope of the invention but merely as illustrations of the presently described embodiment. Many other embodiments of the invention are possible. Therefore, the scope of the invention should be determined, not by the examples given, but by the appended claims and their legal equivalents.

What is claimed is:

1. A clinical trial method for evaluating the efficacy of caries prevention or treatment products comprising the steps of:
a) conducting visual-tactile caries assessment of qualifying human subjects living in populations which exhibit a moderate to high incidence of caries,
b) randomly assigning the qualifying human subjects to two or more test groups, wherein at least one group is an experimental group that is provided an experimental caries prevention or treatment product and at least one group is a control group that is provided a control product,
c) supervising the human subjects brushing their teeth with provided product one or more times per day for a period of about 8 to about 12 months which runs concurrently with and is limited to the school year, and
d) comparing the efficacy of the experimental caries prevention or treatment product to the efficacy of a control product through a follow-up visual-tactile caries assessment of human subjects upon completion of the period of supervised brushing,
wherein throughout the period of supervised brushing, restorative dentistry performed on the human subjects is controlled.

2. The clinical method of claim 1 wherein DMFS or DMFT scoring is used in the visual-tactile caries assessment.

3. The clinical method of claim 2 wherein only teeth or tooth surfaces that contain a clinically relevant carious lesion are scored as decayed.

4. The clinical method of claim 1 wherein the period of supervised brushing is from about 9 to about 12 months that coincides with the school year.

5. The clinical method of claim 1 wherein the human subjects are from about age 5 to about age 15 at the start of the clinical trial.

6. The clinical method of claim 1 wherein the human subjects are from about age 8 to about age 13 at the start of the clinical trial.

7. The clinical method of claim 1 wherein the human subjects are from about age 9 to about age 12 at the start of the clinical trial.

8. The clinical method of claim 1 wherein each test group comprises from about 50 to about 500 qualifying human subjects.

9. The clinical method of claim 1 wherein each test group comprises from about 100 to about 300 qualifying human subjects.

10. The clinical method of claim 1 wherein the visual-tactile assessment is supplemented by bitewing radiographs.

11. The clinical method of claim 1 wherein the supervised brushing occurs two or more times per day.

12. The clinical method of claim 11 wherein the control product is a placebo product.
